(19) United States
(12) Patent Application Publication
(10) Pub. No.: US 2010/0152231 A1
(43) Pub. Date: Jun. 17, 2010

(54) Compositions Useful For Treating Irritable Bowel Syndrome

(75) Inventors: Steven B. Landau, Wellesley, MA (US); Suhail Nurbhai, Sudbury, MA (US)

Correspondence Address: MORGAN, LEWIS & BOCKIUS LLP, 1701 MARKET STREET, PHILADELPHIA, PA, 19103-2921, US

(73) Assignee: EDUSA Pharmaceuticals, Inc.

(21) Appl. No.: 12/635,498

(22) Filed: Dec. 10, 2009

Related U.S. Application Data
(63) Continuation of application No. 12/069,439, filed on Feb. 8, 2008, now abandoned.
(60) Provisional application No. 60/901,141, filed on Feb. 12, 2007.

(51) Int. Cl. A61K 31/439; A61P 1/00

(52) U.S. Cl. 514/305

(57) ABSTRACT

The present invention relates to a method of treating irritable bowel syndrome with constipation (IBS-c) or irritable bowel syndrome with alternating constipation and diarrhea (IBS-a) in a subject in need of treatment. The method comprises administering to said subject a therapeutically effective amount of a thieno[3,2-b]pyridine compound of Structural Formula I or a pharmaceutically acceptable salt or N-oxide derivative thereof.

### RELATED APPLICATION

This application claims the benefit of U.S. Provisional Application No. 60/901,141, filed on Feb. 12, 2007. The entire teachings of the above application are incorporated herein by reference.

### BACKGROUND OF THE INVENTION

Irritable bowel syndrome (IBS) is characterized by symptoms of abdominal pain, discomfort and altered bowel function. Although not life threatening, it significantly reduces quality of life with a major impact on the well being of the patient and social cost to the community. IBS affects 10 to 20% of adults and causes alterations in bowel function, present as diarrhea, constipation or alternation between the two. As such, IBS is a condition that includes three main subtypes of presentation: diarrhea predominant (IBS with diarrhea, IBS-d), constipation predominant (IBS with constipation, IBS-c) and alternating (IBS-a).

IBS (e.g., IBS-c and IBS-a) is typically associated with altered visceral sensitivity and altered intestinal motility. Non-pharmacological treatment options for IBS-c include changes in the fiber content of the diet, biofeedback and psychological counseling. These non-pharmacological options have limited effectiveness on the clinical manifestations of IBS-c, and in some instances (e.g., when fiber intake is increased) can have an adverse influence on the symptoms of abdominal pain and bloating associated with IBS-c. Pharmacological treatment options for IBS-c are limited to ZELNORM® (tegaserod maleate), the only approved drug for the treatment of IBS-c in the United States. ZELNORM® is only approved for use in women, and only for short term treatment, thereby limiting its benefits to a specific subpopulation of patients and a specific time period of treatment. In addition, ZELNORM® is only minimally effective. Furthermore, serious adverse consequences of ZELNORM® include clinically significant diarrhea, including hypovolemia, hypotension and syncope. Ischemic colitis and other forms of intestinal ischemia have also been reported in patients receiving ZELNORM® during marketed use, often resulting in hospitalization.

In view of the above, better treatment options for IBS, in particular IBS-c and IBS-a, are needed.

### SUMMARY OF THE INVENTION

The present invention relates to a method of treating irritable bowel syndrome with constipation (IBS-c) or irritable bowel syndrome with alternating constipation and diarrhea (IBS-a) in a subject in need of treatment. The method comprises administering to said subject a therapeutically effective amount of a thieno[3,2-b]pyridine compound of Structural Formula I or a pharmaceutically acceptable salt or N-oxide derivative thereof.

The invention provides a method of treating irritable bowel syndrome with constipation (IBS-c) or irritable bowel syndrome with alternating constipation and diarrhea (IBS-a) in a subject in need of treatment comprising administering to the subject a therapeutically effective amount of a thieno[3,2-b]pyridine derivative compound represented by Structural Formula I:

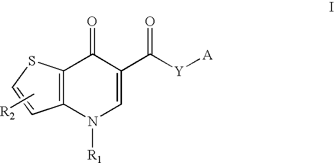

wherein:
R₁ represents hydrogen, a C₁-C₆ alkyl group, a C₂-C₆ alkenyl group, a C₂-C₆ alkynyl group, a C₃-C₈ cycloalkyl group, a C₆-C₁₂ aryl group or a C₇-C₁₈ aralkyl group;
R₂ represents hydrogen, a C₁-C₆ alkyl group, halogen, hydroxyl, a C₁-C₆ alkoxy group, amino, a C₁-C₆ alkylamino group, nitro, mercapto or a C₁-C₆ alkylthio group;
Y represents —O— or

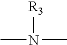

wherein R₃ represents hydrogen or a C₁-C₆ alkyl group; and
A is represented by

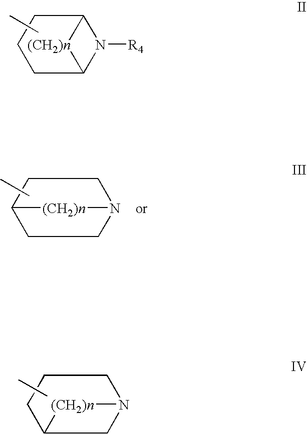

wherein:
n is an integer from 1 to about 4; R₄ represents hydrogen, a C₁-C₆ alkyl group, a C₃-C₈ cycloalkyl group or a C₇-C₁₈ aralkyl group

or a pharmaceutically acceptable salt or N-oxide derivative thereof, wherein the therapeutically effective amount is from greater than 2.4 mg/day to about 8 mg/day.

In specific embodiment of Structural Formula 1, Y represents —O— or

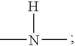

R₁ represents hydrogen, a C₁-C₆ alkyl group, a C₆-C₁₂ aryl group, or a C₇-C₁₈ aralkyl group; R₂ represents hydrogen, a C₁-C₆ alkyl group or halogen; and
A is represented by

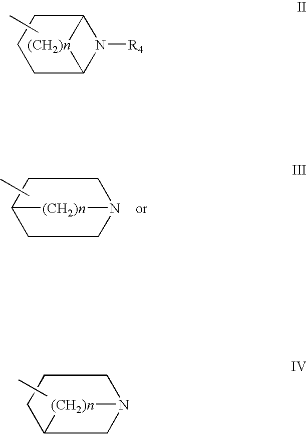

wherein:
   n is 2 or 3; and R₄ represents a C₁-C₆ alkyl group.

In a more specific embodiment, the compound for use in the invention is represented by Structural Formula I, wherein R₁ represents hydrogen or a C₁-C₃ alkyl group; R₂ represents hydrogen, a C₁-C₃ alkyl group or halogen; R₃ represents hydrogen; R₄ represents a C₁-C₃ alkyl group and n is an integer of 2 or 3.

In another specific embodiment, the 5-HT₃ receptor agonist is represented by Structural Formula V:

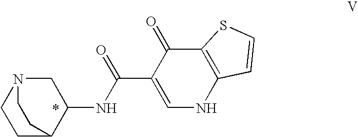

or a pharmaceutically acceptable salt, solvate or hydrate thereof.

In a more specific embodiment, the compound of Structural Formula V has the (R) configuration at the chiral carbon atom which is designated with an asterisk (*). The chemical name of the compound set forth in Structural Formula V having the (R) configuration at the designated chiral carbon is: (R)-N-1-azabicyclo[2.2.2]oct-3-yl-4,7-dihydro-7-oxothieno[3,2-b]pyridine-6-carboxamide.

In a most specific embodiment, the (R)-N-1-azabicyclo[2.2.2]oct-3-yl-4,7-dihydro-7-oxothieno[3,2-b]pyridine-6-carboxamide is in the form of the monohydrochloride, and can be referred to as MKC-733, Dynogen Development Program 733 (DDP733) and pumosetrag (CAS Number: 194093-42-0).

The invention also provides a method of treating IBS-c or IBS-a in a human subject in need of treatment comprising orally administering to the subject a therapeutically effective amount of a compound represented by the following structure:

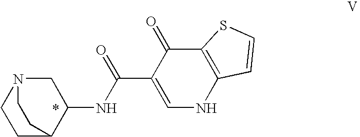

or a pharmaceutically acceptable salt, solvate or hydrate thereof, wherein the therapeutically effect amount is from greater than 2.4 mg/day to about 8 mg/day.

In a specific embodiment, the asterisked carbon atom of the administered compound is in the (R) configuration. In a more specific embodiment, the compound having the (R) configuration is in the form of the monohydrochloride salt.

In a particular embodiment, the subject is suffering from IBS-c.

In one embodiment, the compounds described herein are administered in a single dose. In a specific embodiment, the single dose is about 4.2 mg/day.

In another embodiment, the compounds described herein are administered in multiple doses. For example, administration is two, three, four or more time per day. In a specific embodiment, the multiple doses result in about a 4.2 mg/day dose. In another specific embodiment, the about 4.2 mg/day dose is administered in two divided doses. In a more specific embodiment, the two doses are equivalent. In another specific embodiment, the 4.2 mg/day dose is administered three divided doses. In a more specific embodiment, the three doses are equivalent.

In one embodiment, the compounds described herein are administered coincident with a mealtime of the subject. In a specific embodiment, administration is from about 30 to about 60 minutes before a meal. In a more specific embodiment, the compounds are administered twice a day at about 30 to about 60 minutes prior to a meal. In a most particular embodiment, the compounds described herein are administered three times a day at about 30 to about 60 minutes prior to a meal, for example, prior to breakfast, prior to lunch and prior to dinner of the subject.

In another embodiment, administration is oral.

In another embodiment, the subject is a human. In a specific embodiment, the human subject is a female. In another specific embodiment, the human subject is a male.

In another embodiment, administration of the compounds described herein is in the absence of a laxative.

The invention also provides a method of treating IBS-c or IBS-a in a human in need of treatment comprising orally administering to the subject a therapeutically effective amount of DDP733, wherein the therapeutically effective amount is about 4.2 mg/per day.

In a specific embodiment, the about 4.2 mg/day is administered in three equivalent doses (i.e., about 1.4 mg). In a more specific embodiment, the doses are administered from about 60 minutes to about 30 minutes prior to the breakfast, lunch and dinner of the subject.

The invention further relates to the use of a compound described herein (e.g. a compound of Structural Formula I) for the manufacture of a medicament for treating IBS-c or IBS-a in a subject in need of treatment.

The invention further relates to a pharmaceutical composition useful for treating IBS-c or IBS-a in a subject in need of treatment. The pharmaceutically composition comprises a compound described herein (e.g., a compound of Structural Formula I) and a pharmaceutically acceptable carrier.

### BRIEF DESCRIPTION OF THE DRAWINGS

FIG. 1 is a graph showing the clinical response rates of study subjects (over the entire four week treatment period), determined using the Overall Subject Global Assessment (OSGA), for placebo and various doses of DDP733.

FIG. 2 is a graph showing the week by week response of study subjects receiving 1.4 mg of DDP733 three times a day, and the loss of response (return to level of placebo response) at one week post-treatment.

The foregoing will be apparent from the following more particular description of example embodiments of the invention, as illustrated in the accompanying drawings which like reference characters refer to the same parts throughout the different views. The drawings are not necessarily to scale, emphasis instead being placed upon illustrating embodiments of the present invention.

### DETAILED DESCRIPTION OF THE INVENTION

A description of example embodiments of the invention follows.

The thieno[3,2-b]pyridine compounds of Structural Formula I are described in U.S. Pat. No. 5,352,685, the entire content of which is incorporated herein by reference. The thieno[3,2-b]pyridine derivative compounds of Structural Formula I are known to possess 5-HT₃ receptor agonist activity.

### 5-HT₃ Receptor Agonists

The neurotransmitter serotonin was first discovered in 1948 and has been subsequently the subject of substantial scientific research. Serotonin, also referred to as 5-hydroxytryptamine (5-HT), acts both centrally and peripherally on discrete 5-HT receptors. Currently, at least fourteen subtypes of serotonin receptors are recognized and delineated into seven families, 5-HT₁ through 5-HT₇. These subtypes share sequence homology and display some similarities in their specificity for particular ligands. While these receptors all bind serotonin, they initiate different signaling pathways to perform different functions. For example, serotonin is known to activate submucosal intrinsic nerves via 5-HT_{1P} and 5-HT₄ receptors, resulting in, for example, the initiation of peristaltic and secretory reflexes. However, serotonin is also known to activate extrinsic nerves via 5-HT₃ receptors, resulting in, for example, the initiation and perception of unpleasant bowel sensations, including nausea, bloating and pain. A review of the nomenclature and classification of the 5-HT receptors can be found in Neuropharm., 33: 261-273 (1994) and Pharm. Rev., 46:157-203 (1994).

5-HT₃ receptors are ligand-gated ion channels that are extensively distributed on enteric neurons in the human gastrointestinal tract, as well as other peripheral and central locations. Activation of these channels and the resulting neuronal depolarization has been found to affect the regulation of visceral pain and colonic transit. Antagonism of the 5-HT₃ receptors has the potential to influence sensory and motor function in the gut.

As used herein, 5-HT₃ receptor refers to naturally occurring 5-HT₃ receptors (e.g., mammalian 5-HT₃ receptors (e.g., human (Homo sapiens) 5-HT₃ receptors, murine (e.g., rat, mouse) 5-HT₃ receptors, feline (e.g., cat) 5-HT₃ receptors)) and to proteins having an amino acid sequence which is the same as that of a corresponding naturally occurring 5-HT₃ receptor (e.g., recombinant proteins). The term includes naturally occurring variants, such as polymorphic or allelic variants and splice variants.

As used herein, the term a 5-HT₃ receptor agonist refers to a substance (e.g., a molecule, a compound) which promotes (induces or enhances) at least one function characteristic of a 5-HT₃ receptor. In one embodiment, the 5-HT₃ receptor agonist binds the 5-HT₃ receptor (i.e., is a 5-HT₃ receptor agonist). In certain embodiments, the agonist is a partial agonist. Partial agonist, as used herein, refers to an agonist which no matter how high of a concentration is used, is unable to produce maximal activation of the 5-HT₃ receptor. A 5-HT₃ receptor agonist (e.g., a 5-HT₃ receptor agonist) can be identified and activity assessed by any suitable method. For example, the binding affinity of a 5-HT₃ receptor agonist to the 5-HT₃ receptor can be determined by the ability of the compounds to displace [³H]granisetron from rat cortical membranes (Cappelli et al., J. Med. Chem., 42(9): 1556-1575 (1999)). In addition, the agonist activity of the compounds can be assessed in vitro on, for example, the 5-HT₃ receptor-dependent [¹⁴C]guanidinium uptake in NG 108-15 cells as described in Cappelli et al.

The thieno[3,2-b]pyridine derivative compounds suitable for use in the present invention are represented by Structural Formula I:

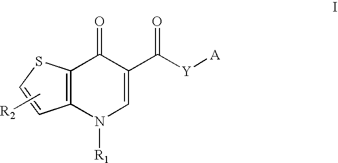

wherein:
R₁ represents hydrogen, a C₁-C₆ alkyl group, a C₂-C₆ alkenyl group, a C₂-C₆ alkynyl group, a C₃-C₈ cycloalkyl group, a C₆-C₁₂ aryl group or a C₇-C₁₈ aralkyl group;
R₂ represents hydrogen, a C₁-C₆ alkyl group, halogen, hydroxyl, a C₁-C₆ alkoxy group, amino, a C₁-C₆ alkylamino group, nitro, mercapto or a C₁-C₆ alkylthio group;
Y represents —O— or

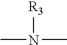

wherein R₃ represents hydrogen or a C₁-C₆ alkyl group; and
A is represented by

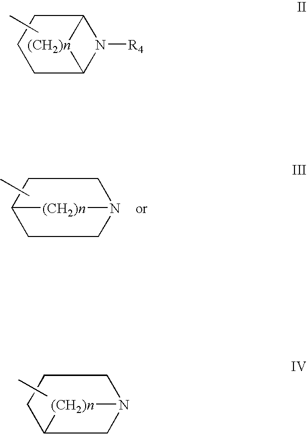

wherein:
n is an integer from 1 to about 4; R₄ represents hydrogen, a C₁-C₆ alkyl group, a C₃-C₈ cycloalkyl group or a C₇-C₁₈ aralkyl group
   or a pharmaceutically acceptable salt thereof.

It is understood that when R₁ of Structural Formula I is hydrogen, compounds having the tautomeric form represented by Structural Formula IA are included within the definition of Structural Formula I.

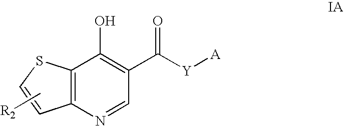

Likewise, it is understood that Structural Formula IA includes the tautomeric form represented by Structural Formula I when R₁ is hydrogen.

In one embodiment, the 5-HT₃ receptor agonist represented by Structural Formula I can be N-oxide derivatives.

In another embodiment of Structural Formula I, Y represents —O— or

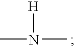

R₁ represents hydrogen, a C₁-C₆ alkyl group, a C₆-C₁₂ aryl group, or a C₇-C₁₈ aralkyl group; R₂ represents hydrogen, a C₁-C₆ alkyl group or halogen; and
A is represented by

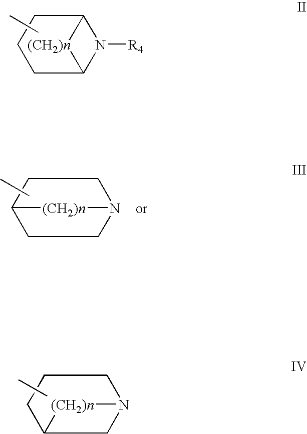

wherein:
   n is 2 or 3; and R₄ represents a C₁-C₆ alkyl group.

In a particular embodiment, the 5-HT₃ receptor agonist is represented by Structural Formula I, wherein R₁ represents hydrogen or a C₁-C₃ alkyl group; R₂ represents hydrogen, a C₁-C₃ alkyl group or halogen; R₃ represents hydrogen; R₄ represents a C₁-C₃ alkyl group and n is an integer of 2 or 3.

In a more particularly embodiment, the 5-HT₃ receptor agonist is represented by Structural Formula V:

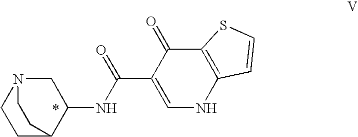

or a pharmaceutically acceptable salt, solvate or hydrate thereof.

In yet another embodiment, the compound represented by Structural Formula V is an N-oxide derivative.

In a most particularly embodiment, the compound of Structural Formula V has the (R) configuration at the chiral carbon atom which is designated with an asterisk (*). The chemical name of the compound set forth in Structural Formula V having the (R) configuration at the designated chiral carbon is: (R)-N-1-azabicyclo[2.2.2]oct-3-yl-4,7-dihydro-7-oxothieno[3,2-b]pyridine-6-carboxamide. When the compound is in the form of the monohydrochloride, it is known as MKC-733, Dynogen Development Program 733 (DDP733) and pumosetrag (CAS Number: 194093-42-0). When the compound of Structural Formula V has the (S) configuration at the chiral carbon atom designated with an asterisk (*), the chemical name is (S)-N-1-azabicyclo[2.2.2]oct-3-yl-4,7-dihydro-7-oxothieno[3,2-b]pyridine-6-carboxamide.

It is understood that Structural Formula V includes the tautomeric form depicted by Structural Formula VA:

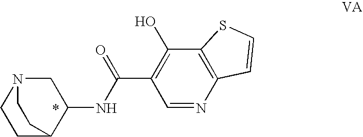

Likewise, it is understood that Structural Formula VA includes the tautomeric form represented by Structural Formula V.

For example, when Structural Formula V has the (R) configuration at the designated chiral carbon the compound is referred to as: (R)-N-1-azabicyclo[2.2.2]oct-3-yl-4,7-dihydro-7-oxothieno[3,2-b]pyridine-6-carboxamide which is understood to include the tautomeric form: (R)-N-1-azabicyclo[2.2.2]oct-3-yl)-7-hydroxythieno[3,2-b]pyridine-6-carboxamide.

Likewise, when Structural Formula VA has the (R) configuration at the designated chiral carbon the compound is referred to as: (R)-N-1-azabicyclo[2.2.2]oct-3-yl)-7-hydroxythieno[3,2-b]pyridine-6-carboxamide, which is understood to include the tautomeric form: (R)-N-1-azabicyclo[2.2.2]oct-3-yl-4,7-dihydro-7-oxothieno[3,2-b]pyridine-6-carboxamide.

As used herein, the term “compound” is intended to include any solvates, hydrates or polymorphs thereof. Thus, it is to be understood that when any compound is referred to by name and structure, solvates, hydrates and polymorphs thereof are included.

### Irritable Bowel Syndrome

IBS is a functional bowel disorder in which abdominal discomfort or pain is associated with defecation or change in bowel habit and with features of disordered defecation. The Rome III Diagnostic Criteria* for IBS is as follow:

Recurrent abdominal pain or discomfort** at least 3 days per month in the last 3 months associated with 2 or more of the following:
a) Improvement with defecation; and/or
b) Onset associated with a change in frequency of stool; and/or
c) Onset associated with a change in form (appearance) of stool.
*Criteria fulfilled for the last 3 months with symptom onset at least 6 months prior to diagnosis.
**“Discomfort” means an uncomfortable sensation not described as pain.
The following symptoms cumulatively support the diagnosis of IBS:
abnormal stool frequency (for research purposes “abnormal” can be defined as >3/day and <3/week);
abnormal stool form (lumpy/hard or loose/watery stool);
abnormal stool passage (straining, urgency, or feeling of incomplete evacuation);
passage of mucus;
bloating or feeling of abdominal distension.

To support a diagnosis of IBS-c, the abnormal stool frequency is fewer than three bowel movements per week (0-2); abnormal stool form is lumpy/hard; and abnormal stool passage includes straining or feeling of incomplete evacuation.

To support a diagnosis of IBS-a, the subject has IBS with alternating constipation and diarrhea.

Subjects with IBS consistently exhibit visceral hypersensitivity. It is believed that the pain associated with IBS is primarily a result of this hypersensitivity of the visceral afferent nervous system. For example, patients and controls were evaluated for their pain thresholds in response to progressive distension of the sigmoid colon induced by a balloon. At the same volume of distension, the patients reported higher pain scores compared to controls. This finding has been reproduced in many studies and with the introduction of the barostat, a computerized distension device, the distension procedures have been standardized. Two concepts of visceral hypersensitivity, hyperalgesia and allodynia, have been introduced. More specifically, hyperalgesia refers to the situation in which normal visceral sensations are experienced at lower intraluminal volumes. While for a finding of allodynia, pain or discomfort is experienced at volumes usually producing normal internal sensations (see, for example, Mayer E. A. and Gebhart, G. F., Basic and Clinical Aspects of Chronic Abdominal Pain, Vol 9, 1st ed. Amsterdam: Elsevier, 1993:3-28).

As such, IBS is a functional bowel disorder in which abdominal pain or discomfort is associated with defecation or a change in bowel habit. Therefore, IBS has elements of an intestinal motility disorder, a visceral sensation disorder, and a central nervous system disorder. No physiological mechanism unique to IBS has been identified. In some cases, the same mechanisms that cause occasional abdominal discomfort in healthy individuals operate to produce the symptoms of IBS. The symptoms of IBS may therefore be a product of quantitative differences in the motor reactivity of the intestinal tract, and increased sensitivity to stimuli or spontaneous contractions.

As such, IBS-c and IBS-a are disorders having a constellation of symptoms. Disorders with such a characterization make identification of suitable medicaments and/or suitable doses of a medicament, which can provide on overall benefit to the patient (rather than addressing specific symptoms), difficult. Further, there is no biomarker test which can predict a compound or dose which will be efficacious in relieving the symptoms of IBS. Therefore, although a compound may have demonstrated attributes which could indicate benefit for some features associated with IBS, this cannot predict efficacy in the overall alleviation of IBS. For example, fiber and bulking agents are used to treat constipation, but their efficacy in IBS can lead to worsening of the symptoms.

As used herein, therapeutically effective amount refers to an amount sufficient to elicit the desired biological response. In the present invention the desired biological response can be an overall improvement in the condition being treated. The overall improvement can be associated with improvement in individual symptoms. For example, a desired biological response can include improvement (complete or partial reduction) of at least one symptom associated with IBS-c or IBS-a. For example, a reduction in the pain or discomfort associated with IBS-c or IBS-a is considered a desired biological response. As with any treatment, particularly treatment of a multi-symptom disorder, for example, IBS-c and IBS-a, it is advantageous to treat as many disorder-related symptoms which the subject experiences. For example, when the subject is being treated for IBS-c or IBS-a, a reduction in the pain or discomfort associated with IBS and a reduction in at least one other symptom of IBS selected from abnormal stool frequency, abnormal stool form, abnormal stool passage, passage of mucus and bloating or feeling of abdominal distension is preferred. It is most preferred, however, that the subject being treated for IBS-c or IBS-a experience an overall relief from the symptoms of the disorder. Such overall relief can be assessed using a Subject Global Assessment of Overall Relief (OSGA).

Subject, as used herein, refers to animals such as mammals, including, but not limited to, primates (e.g., humans), cows, sheep, goats, horses, pigs, dogs, cats, rabbits, guinea pigs, rats, mice or other bovine, ovine, equine, canine, feline, rodent or murine species. In one embodiment, the subject is a human. In a particular embodiment, the human is a female. In another particular embodiment, the human is a male.

It is understood that the length of time the subject has been suffering from IBS-c or IBS-a can vary. In a one embodiment, the subject can be recently afflicted with IBS-c or IBS-a, such as within the last 12 months. In another embodiment, the subject has suffered with IBS-c or IBS-a for one year or more, for example, about 1 year, about 2 years, about 3 years, about 4 years, about 5 years or more. In certain embodiments, the subject has suffered from IBS-c or IBS-a more than half their lifetime (e.g, a 50 year old subject has suffered for more than 25 years). In a specific embodiment, onset of the IBS-c or IBS-a in the subject occurred during adulthood (adult onset). In another specific embodiment, onset of the IBS-c or IBS-a in the subject occurred prior to adulthood (pre-adult onset).

### Modes of Administration

The compounds for use in the method of the invention can be formulated for oral, transdermal, sublingual, buccal, parenteral, rectal, intranasal, intrabronchial or intrapulmonary administration. Oral administration is preferred. For oral administration the compounds can be of the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets can be coated using suitable methods and coating materials such as OPADRY® film coating systems available from Colorcon, West Point, Pa. (e.g., OPADRY® OY Type, OY-C Type, Organic Enteric OY-P Type, Aqueous Enteric OY-A Type, OY-PM Type and OPADRY® White, 32K18400).

In a particular embodiment, the oral form is a tablet containing DDP733 and the inactive ingredients mannitol, corn starch, microcrystalline cellulose, colloidal silicon dioxide, polyvinyl pyrrolidone, talc, and magnesium stearate, which are coated with an OPADRY® film coating. For example, a 1.4 mg dose of DDP733 can include: 1.4 mg of DDP733; Mannitol 89 mg: Corn starch 24.7 mg; Microcrystalline cellulose 6.8 mg; Colloidal silicon dioxide 0.7 mg; Polyvinyl pyrrolidone 2.7 mg; Talc 0.7; Magnesium stearate 4.0 mg; and Opadry white 6.5 mg.

Liquid preparation for oral administration can be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

For buccal administration, the compounds for use in the method of the invention can be in the form of tablets or lozenges formulated in a conventional manner.

For parenteral administration, the compounds for use in the method of the invention can be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents can be used.

For rectal administration, the compounds for use in the method of the invention can be in the form of suppositories or enemas.

For sublingual administration, tablets can be formulated in conventional manner.

For intranasal, intrabronchial or intrapulmonary administration, conventional formulations can be employed.

Further, the compounds for use in the method of the invention can be formulated in a sustained release preparation. For example, the compounds can be formulated with a suitable polymer or hydrophobic material which provides sustained and/or controlled release properties to the active agent compound. As such, the compounds for use the method of the invention can be administered in the form of microparticles for example, by injection or in the form of wafers or discs by implantation.

### Dosing

The therapeutically effect amount of the compound of Formula I can be in the range of greater than 2.4 mg/day to about 8.0 mg/day. The dose can be administered from 1 to 3 or more times per day. When multiple dosages are used, the amount of each dosage can be the same or different.

The range of greater than 2.4 mg/day to about 8 mg/day includes all incremental doses within the range. For example, about 2.5 mg/day, about 2.6 mg/day, about 2.7 mg/day, about 2.8 mg/day, about 2.9 mg/day, about 3.0 mg/day, about 3.1 mg/day, about 3.2 mg/day, about 3.3 mg/day, about 3.4 mg/day, about 3.5 mg/day, about 3.6 mg/day, about 3.7 mg/day, about 3.8 mg/day, about 3.9 mg/day, about 4.0 mg/day, about 4.1 mg/day, about 4.2 mg/day, about 4.3 mg/day, about 4.4 mg/day, about 4.5 mg/day, about 4.6 mg/day, about 4.7 mg/day, about 4.8 mg/day, about 4.9 mg/day, about 5.0 mg/day, about 5.1 mg/day, about 5.2 mg/day, about 5.3 mg/day, about 5.4 mg/day, about 5.5 mg/day, about 5.6 mg/day, about 5.7 mg/day, about 5.8 mg/day, about 5.9 mg/day, about 6.0 mg/day, about 6.1 mg/day, about 6.2 mg/day, about 6.3 mg/day, about 6.4 mg/day, about 6.5 mg/day, about 6.6 mg/day, about 6.7 mg/day, about 6.8 mg/day, about 6.9 mg/day, about 7.0 mg/day, about 7.1 mg/day, about 7.2 mg/day, about 7.3 mg/day, about 7.4 mg/day, about 7.5 mg/day, about 7.6 mg/day, about 7.7 mg/day, about 7.8 mg/day, about 7.9 mg/day and about 8.0 mg/day.

Other dose ranges suitable for use in the invention include from: about 2.5 mg/day to about 8.0 mg/day, about 2.6 mg/day to about 8.0 mg/day, about 2.7 mg/day to about 8.0 mg/day, about 2.9 mg/day to about 8.0 mg/day, about 3.0 mg/day to about 8.0 mg/day, about 3.1 mg/day to about 8.0 mg/day, about 3.2 mg/day to about 8.0 mg/day, about 3.3 mg/day to about 8.0 mg/day, about 3.4 mg/day to about 8.0 mg/day, about 3.5 mg/day to about 8.0 mg/day, about 3.6 mg/day to about 8.0 mg/day, about 3.7 mg/day to about 8.0 mg/day, about 3.8 mg/day to about 8.0 mg/day, about 3.9 mg/day to about 8.0 mg/day, about 4.0 mg/day to about 8.0 mg/day, about 4.1 mg/day to about 8.0 mg/day, about 4.2 mg/day to about 8.0 mg/day, about 4.3 mg/day to about 8.0 mg/day, about 4.4 mg/day to about 8.0 mg/day, about 4.5 mg/day to about 8.0 mg/day, about 4.6 mg/day to about 8.0 mg/day, about 4.7 mg/day to about 8.0 mg/day, about 4.8 mg/day to about 8.0 mg/day, about 4.9 mg/day to about 8.0 mg/day, about 5.0 mg/day to about 8.0 mg/day, about 5.1 mg/day to about 8.0 mg/day, about 5.2 mg/day to about 8.0 mg/day, about 5.3 mg/day to about 8.0 mg/day, about 5.4 mg/day to about 8.0 mg/day, about 5.5 mg/day to about 8.0 mg/day, about 5.6 mg/day to about 8.0 mg/day, about 5.7 mg/day to about 8.0 mg/day, about 5.8 mg/day to about 8.0 mg/day, about 5.9 mg/day to about 8.0 mg/day, about 6.0 mg/day to about 8.0 mg/day, about 6.1 mg/day to about 8.0 mg/day, about 6.2 mg/day to about 8.0 mg/day, about 6.3 mg/day to about 8.0 mg/day, about 6.4 mg/day to about 8.0 mg/day, about 6.5 mg/day to about 8.0 mg/day, about 6.6 mg/day to about 8.0 mg/day, about 6.7 mg/day to about 8.0 mg/day, about 6.8 mg/day to about 8.0 mg/day, about 6.9 mg/day to about 8.0 mg/day, about 7.0 mg/day to about 8.0 mg/day, about 7.1 mg/day to about 8.0 mg/day, about 7.2 mg/day to about 8.0 mg/day, about 7.3 mg/day to about 8.0 mg/day, about 7.4 mg/day to about 8.0 mg/day, about 7.5 mg/day to about 8.0 mg/day, about 7.6 mg/day to about 8.0 mg/day, about 7.7 mg/day to about 8.0 mg/day, about 7.8 mg/day to about 8.0 mg/day, and about 7.9 mg/day to about 8.0 mg/day.

Additional dose ranges include from: about 2.5 mg/day to about 8.0 mg/day, about 2.5 mg/day to about 7.9 mg/day, about 2.5 mg/day to about 7.8 mg/day, about 2.5 mg/day to about 7.7 mg/day, about 2.5 mg/day to about 7.6 mg/day, about 2.5 mg/day to about 7.5 mg/day, about 2.5 mg/day to about 7.4 mg/day, about 2.5 mg/day to about 7.3 mg/day, about 2.5 mg/day to about 7.2 mg/day, about 2.5 mg/day to about 7.1 mg/day, about 2.5 mg/day to about 7.0 mg/day, about 2.5 mg/day to about 6.9 mg/day, about 2.5 mg/day to about 6.8 mg/day, about 2.5 mg/day to about 6.7 mg/day, about 2.5 mg/day to about 6.6 mg/day, about 2.5 mg/day to about 6.5 mg/day, about 2.5 mg/day to about 6.4 mg/day, about 2.5 mg/day to about 6.3 mg/day, about 2.5 mg/day to about 6.2 mg/day, about 2.5 mg/day to about 6.1 mg/day, about 2.5 mg/day to about 6.0 mg/day, about 2.5 mg/day to about 5.9 mg/day, about 2.5 mg/day to about 5.8 mg/day, about 2.5 mg/day to about 5.7 mg/day, about 2.5 mg/day to about 5.6 mg/day, about 2.5 mg/day to about 5.5 mg/day, about 2.5 mg/day to about 5.4 mg/day, about 2.5 mg/day to about 5.3 mg/day, about 2.5 mg/day to about 5.2 mg/day, about 2.5 mg/day to about 5.1 mg/day, about 2.5 mg/day to about 5.0 mg/day, about 2.5 mg/day to about 4.9 mg/day, about 2.5 mg/day to about 4.8 mg/day about 2.5 mg/day to about 4.7 mg/day, about 2.5 mg/day to about 4.6 mg/day, about 2.5 mg/day to about 4.5 mg/day, about 2.5 mg to about 4.4 mg, about 2.5 mg to about 4.3 mg, about 2.5 mg to about 4.2 mg about 2.5 mg/day to about 4.1 mg/day, about 2.5 mg/day to about 4.0 mg/day, about 2.5 mg/day to about 3.9 mg/day, about 2.5 mg/day to about 3.8 mg/day, about 2.5 mg/day to about 3.7 mg/day, about 2.5 mg/day to about 3.6 mg/day, about 2.5 mg/day to about 3.5 mg/day, about 2.5 mg/day to about 3.4 mg/day, about 2.5 mg/day to about 3.3 mg/day, about 2.5 mg/day to about 3.2 mg/day, about 2.5 mg/day to about 3.1 mg/day, about 2.5 mg/day to about 3.0 mg/day, about 2.5 mg/day to about 2.9 mg/day, about 2.5 mg/day to about 2.8 mg/day, about 2.5 mg/day to about 2.7 mg/day, and about 2.5 g/day to about 2.6 mg/day.

Other suitable dosing ranges include, for example, from about 2.5 mg/day to about 7.9 mg/day, about 2.6 mg/day to about 7.8 mg/day, about 2.7 mg/day to about 7.7 mg/day, about 2.8 mg/day to about 7.6 mg/day, about 2.9 mg/day to about 7.5 mg/day, about 3.0 mg/day to about 7.4 mg/day, about 3.1 mg/day to about 7.3 mg/day, about 3.2 mg/day to about 7.2 mg/day, about 3.3 mg/day to about 7.1 mg/day, about 3.4 mg/day to about 7.0 mg/day, about 3.5 mg/day to about 6.9 mg/day, about 3.6 mg/day to about 6.8 mg/day, about 3.7 mg/day to about 6.7 mg/day, about 3.8 mg/day to about 6.6 mg/day, about 3.9 mg/day to about 6.5 mg/day, about 4.0 mg/day to about 6.4 mg/day, about 4.1 mg/day to about 6.3 mg/day, about 4.2 mg/day to about 6.2 mg/day, about 4.3 mg/day to about 6.1 mg/day, about 4.4 mg/day to a bout 6.0 mg/day, about 4.5 mg/day to about 5.9 mg/day, about 4.6 mg/day to about 5.8 mg/day, about 4.7 mg/day to about 5.7 mg/day, about 4.8 mg/day to about 5.6 mg/day, about 4.9 mg/day to about 5.5 mg/day, about 5.0 mg/day to about 5.4 mg/day, and about 5.1 mg/day to about 5.3 mg/day.

It is understood that each of these doses can be administered from one to three or more times per day. When multiple dosages are used, the amount of each dosage can be the same or different. For example, when the dose per day is about 4.2 mg, this amount can be administered in a single dose, in two doses of equal or different amounts or in three doses of equal or different amounts. A preferred dose is 4.2 mg/day which can be administered in three doses of equal amount (i.e., 1.4 mg)

The dose of the compound of Formula I can be administered at equally spaced intervals in a 24 hour day (e.g., 3 times a day at every 8 hours) or at varying intervals of time during a 24 hour day.

Further, the dose of the compound of Formula I can be administered coincident with the subject's mealtimes (e.g., breakfast, lunch and dinner). By coincident with mealtimes is meant at most about one hour before a meal, for example from about 30 minutes to about 60 minutes before a meal, or one hour after a meal, for example from about 30 minutes to about 60 minutes after a meal. For example, a suitable dosing regimen can be three times a day, 30 to 60 minutes before breakfast, lunch and dinner.

In one embodiment, the compound of Formula I is administered in the absence of a laxative, In the absence of a laxative means that treatment of the subject is conducted without the use of a laxative to assist with the symptom of constipation.

The compounds for use in the method of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

For the compounds of Formula I, each dosage can typically contain from greater than 2.4 mg to about 8.0 mg. In addition, all of the ranges and specific doses listed above can be present in a unit dosage form. In a preferred embodiment, the compound of Formula I is DDP733 and is present in the unit dosage form at about 4.2 mg in a single dose or in 2, 3 or more doses, which are the same or different in amount.

The invention further includes a kit for treating IBS-c or IBS-a. The kit comprises at a compound of Formula I and an instruction insert for administering the compound according to the method of the invention. In a preferred embodiment, the kit includes greater than 2.4 mg to about 8.0 mg of the compound of Formula I. In a more preferred embodiment, the compound of Formula I is DDP733. In a most preferred embodiment, the kit provides a dose of 4.2 mg and is for a daily dose.

As used herein, the term pharmaceutically acceptable salt refers to a salt of the administered compounds prepared from pharmaceutically acceptable non-toxic acids including inorganic acids, organic acids, solvates, hydrates, or clathrates thereof. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like.

### Clinical Trial Results

The Clinical Trial reported herein was a Phase 2a, Randomized, Double-Blind, Placebo-Controlled Study. In the Study, 91 males and females with IBS-c were administered doses of DDP733 (0.2, 0.5, 0.8 and 1.4 mg) and placebo, three times a day for 28 days (4 weeks).

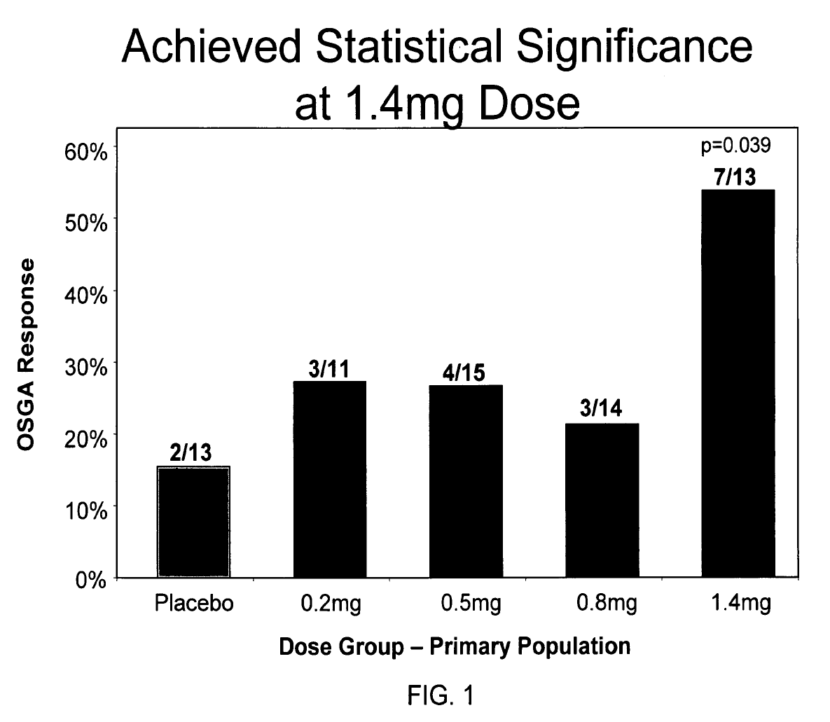

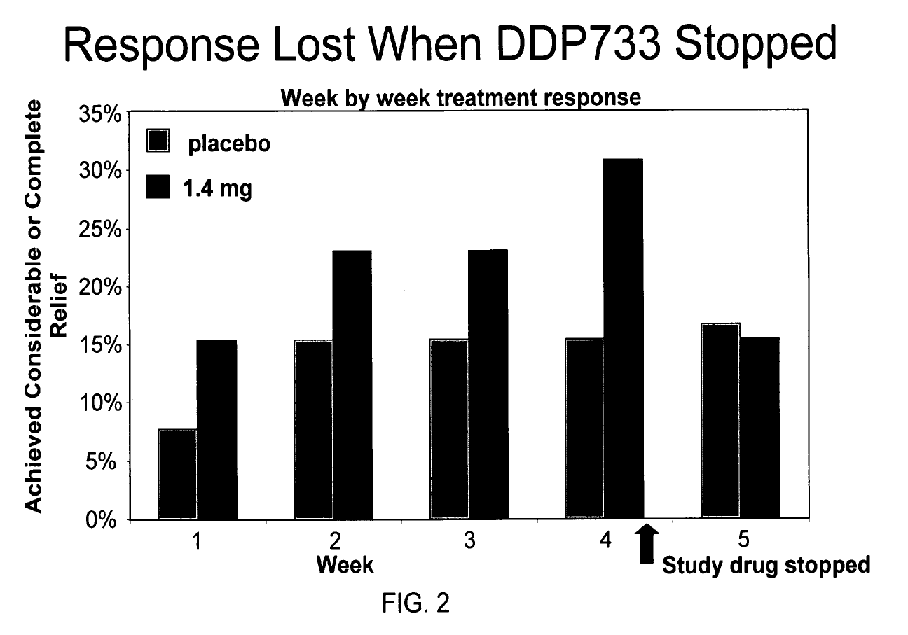

The results of the clinical trial of the present invention show that a dose in the range of greater than 2.4 mg per day up to about 8 mg per day (i.e., 1.4 mg three times a day=4.2 mg/day) achieved statistical significance on the clinical endpoint, overall symptom relief, as determined using Subject Global Assessment of overall relief (OSGA) (FIG. 1). Notably, FIG. 1 shows that 54% of the subjects receiving the 1.4 mg dose three times a day reported overall symptom relief, as compared to about 15% of subject receiving placebo. Importantly, the OSGA clinical response seen with the 1.4 mg three times a day dose, was lost (i.e., returned to the level of placebo) at one week post-treatment (FIG. 2). This loss indicates that the response observed was due to treatment with DDP733.

The OSGA is an accepted endpoint in clinical assessment of IBS-c and results of OSGA were used to support the FDA approval of ZELNORM®, the only drug currently approved for the treatment of IBS-c.

Study Details:
Phase 2a, randomized, double-blinded, placebo controlled parallel group design
91 males and females with IBS-c
7 week study with 4 weeks of treatment
Five treatment groups:
   DDP733: 0.2 mg, 0.5 mg, 0.8 mg or 1.4 mg three times a day (approximately 30 to 60 minutes before breakfast, lunch and dinner)
   PLACEBO: Three times a day (approximately 30 to 60 minutes before breakfast, lunch and dinner).

Objectives
Assess effect on relief of IBS-c using symptom-based assessment including the following weekly questions: Overall response (OSGA); Abdominal discomfort/pain; Constipation; and Satisfaction with bowel habit.
Assess effect on pharmacodynamic measure (gastrointestinal transit) at baseline and week 4 using radio-opaque markers.
Establish safety profile.

Key Subject Inclusion Criteria:
1. Male or female from 18 to 65 years of age, inclusive
2. IBS-c for at least three months prior to randomization as defined by the subject's response to the criteria below adapted from the Rome II Modular Questionnaire:
   a. Subject experiences discomfort or pain in the abdomen during at least 3 weeks (at least one day in each week) in the last 3 months.
   b. Subject experiences one or more of the following:
      i. Discomfort or pain that gets better or stops after a bowel movement
      ii. Change in the usual number of bowel movements (either more or fewer) when the discomfort or pain starts
      iii. Softer or harder stools than usual when the discomfort or pain starts
   c. Patient experiences two or more of the following at least one-fourth (¼) of the occasions or days in the last three months:
      i. Fewer than three bowel movements per week (0-2)
      ii. Hard or lumpy stools
      iii. Straining during a bowel movement
   d. Patient experiences none of the following more than one-fourth (¼) of the occasions or days in the last three months:
      i. More than three bowel movements a day (4 or more)
      ii. Loose, mushy or watery stools
      iii. Having to rush to the toilet to have a bowel movement
3. Completion of at least six days of daily diary assessments in the week of screening prior to Visit 3 with a documented average stool consistency score of ≦2.5. Stool consistency was rated as 1=very hard, 2=hard, 3=formed, 4=loose, 5=watery.

Clinical Endpoints Measurements:

Clinical Symptoms were assessed using the following weekly questions:

(1) Overall response (OSGA);

(2) Abdominal discomfort/pain;

(3) Constipation; and

(4) Satisfaction with bowel habit.

| VARIABLE | QUESTION | RESPONSE SCALE |
|---|---|---|
| (1) Overall | “Please consider how you felt | 1 = Completely relieved; |
| response | the past week in regard to your | 2 = Considerably |
| | IBS, in particular your overall | relieved; |
| | well being and symptoms of | 3 = Somewhat relieved; |
| | abdominal discomfort, pain | 4 = Unchanged; and |
| | and altered bowel habit. | 5 = Worse |
| | Compared to the way you | |
| | usually felt before entering the | |
| | study, how would you rate | |
| | your relief of symptoms | |
| | during the past week?” | |
| (2) | “How bothersome was your | 0 = not at all; |
| Abdominal | abdominal discomfort and | 1 = hardly; |
| discomfort/ | pain over the past week?” | 2 = somewhat; |
| pain | | 3 = moderately; |
| | | 4 = a good deal; |
| | | 5 = a great deal; |
| | | 6 = a very great deal |
| (3) | “How bothersome was your | 0 = not at all; |
| Constipation | constipation over the past | 1 = hardly; |
| | week?” | 2 = somewhat; |
| | | 3 = moderately; |
| | | 4 = a good deal; |
| | | 5 = a great deal; |
| | | 6 = a very great deal |
| (4) | “How satisfied were you with | 1 = satisfied; |
| Satisfaction | your bowel habits over the | 2 = somewhat satisfied; |
| with bowel | past week?” | 3 = somewhat |
| habit | | dissatisfied; 4 = very |
| | | dissatisfied |

### Results:

Statistical significance (using the Pearson chi squared test) was achieved for the clinical endpoint of subject global assessment of relief overall (Overall Subject Global Assessment-OSGA, Questionnaire 1) in the treatment group receiving 1.4 mg of DDP733, three times a day. Subjects were defined as Overall SGA responders, prior to the study, if:
1. Somewhat relieved or better at 100% of SGAs (Response 3 at all weekly time points), or considerably/completely relieved for at least 50% of SGAs (Response 1 or 2 for at least 2 of the 4 weekly timepoints) or considerably/completely relieved at Week 4 (Response 1 or 2 at Week AND
2. No more than 5 days of laxative use excluding bulk-forming laxatives over the 4-week treatment period.

Overall symptom relief was achieved in 54% of the IBS-c subjects administered the 4.2 mg/day dose (1.4 mg three times a day) versus 15% for placebo. In other words, 54% of the subjects administered 4.2 mg/day fell within the definition of overall SGA responder. The results of this clinical endpoint are presented graphically in FIG. 1.

Notably, FIG. 2 which graphically depicts the week by week OSGA clinical response of study subjects receiving 1.4 mg of DDP733 three times a day, shows a loss of response (return to level of placebo response) at one week post-treatment. This loss indicates that the response observed was due to treatment with DDP733.

It is noted that in the study group showing overall symptom relief (4.2 mg/day) that relief was achieved in all responders without the use of laxatives and that no responders reported any diarrhea during the treatment period.

The other weekly subject global assessments (abdominal discomfort/pain, constipation and satisfaction with bowel habit, Questionnaires 2, 3 and 4) showed consistent trends supporting the OSGA. For example, a consistent pattern of improvement was seen for the 4.2 mg/day dose, but not for placebo.

In addition, for overall responders within the 1.4 mg, 3 times a day treatment group, a decrease in weekly pain response was observed (Variable 2 in Table). This decrease in pain appears to be a result of the pharmacological action of the DDP733, because other specific features of IBS, for example, constipation and satisfaction with bowel habit also improved on treatment in the group. Such a decrease in pain in unexpected, because the 5-HT₃ agonist action of DDP733 might be expected to cause an increase in pain since drugs having the opposite pharmacological action (5-HT₃ receptor antagonists) are known to reduce the perception of pain.

### Pharmacodynamic Measurements:

Pharmacodynamic measurements were conducted to assess colonic transit time. Each subject was instructed to ingest three capsules containing radio-opaque markers (SITZMARKS®) on three consecutive days. In many subjects, the radio-opaque markers had not been ingested according to the study protocol and no conclusions could be draw from the assessment.

### Safety Measurements:

Safety measurements included monitoring of vital signs and adverse events, clinical laboratory testing and performance of electrocardiograms (ECGs).

Drug related adverse events (nausea, pruritis and rash) were mainly mild-moderate, transient in nature and required no intervention. These was no pattern of lab or ECG abnormalities.

While this invention has been particularly shown and described with references to example embodiments thereof, it will be understood by those skilled in the art that various changes in form and details may be made therein without departing from the scope of the invention encompassed by the appended claims.

What is claimed is:

1. A method of treating IBS-c or IBS-a in a human subject in need thereof comprising orally administering to said subject a therapeutically effective amount of a compound represented by the following structure: 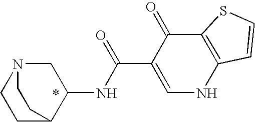
or a pharmaceutically acceptable salt, solvate or hydrate thereof, wherein the therapeutically effective amount ranges from greater than 2.4 mg/per day to about 8.0 mg/day.

2. The method of claim 1, wherein the subject suffers from IBS-c.

3. The method of claim 1 or 2, wherein the asterisked carbon atom is in the (R) configuration.

4. The method of claim 3, wherein the compound is in the form of the monohydrochloride salt.

5. The method of claim 1 or 2, wherein the compound is administered in a single dose.

6. The method of claim 1 or 2, wherein the compound in administered in multiple dosages.

7. The method of claim 6, wherein the compound is administered in equivalent doses twice a day.

8. The method of claim 6, wherein the compound is administered in equivalent doses three times a day.

9. The method of claim 8, wherein the compound is administered coincident with the breakfast, lunch and dinner meals of the subject.

10. The method of claim 5, wherein about 4.2 mg per day is administered.

11. The method of claim 6, wherein about 4.2 mg per day is administered.

12. The method of claim 7, wherein about 4.2 mg per day is administered.

13. The method of claim 8, wherein about 4.2 mg per day is administered.

14. The method of claim 9, wherein about 4.2 mg per day is administered.

15. A method of treating irritable bowel syndrome with constipation (IBS-c) in a human in need of treatment comprising orally administering to the subject a therapeutically effective amount of DDP-733, wherein the therapeutically effective amount is about 4.2 mg/per day.

16. The method of claim 15, wherein the about 4.2 mg/day dose is administered in three equivalent doses.

17. The method of claim 16, wherein each dose is administered from about 60 minutes to about 30 minutes prior to the breakfast, lunch and dinner of the subject.
